(19) United States
(12) Patent Application Publication
(10) Pub. No.: US 2012/0277544 A1
(43) Pub. Date: Nov. 1, 2012

(54) BIODEGRADABLE INSERTION GUIDE FOR THE INSERTION OF A MEDICAL DEVICE

(75) Inventors: Brian C. A. Fernandes, Roseville, MN (US); Frans L. H. Gielen, Eckelrade (NL); Peter Appenrodt, Bermen (DE)

(73) Assignee: MEDTRONIC, INC., Minneapolis, MN (US)

(21) Appl. No.: 13/450,652

(22) Filed: Apr. 19, 2012

Related U.S. Application Data
(60) Provisional application No. 61/479,893, filed on Apr. 28, 2011.

(51) Int. Cl. A61B 19/00; A61B 5/00; A61M 25/00

(52) U.S. Cl. 600/300; 604/528; 606/129

(57) ABSTRACT

The present invention includes an insertion guide configured to be inserted in combination with a stylet wherein the insertion guide is left in the brain after the stylet is removed. The insertion guide then provides a path way for the stimulation lead, catheter, or other medical device to be placed into the brain to allow for the application of stimulation or therapeutic fluids to be administered. The insertion guide is further made of biodegradable material such that, after the lead is inserted through the insertion guide, the material forming the insertion guide biodegrades and is absorbed by the body.

### RELATED APPLICATION

This application claims the benefit of the filing date of provisional U.S. Application Ser. No. 61/479,893, filed Apr. 28, 2011.

### TECHNICAL FIELD

The disclosure relates to medical systems, and, more particularly, medical systems for guidance of an implantable medical device to a target.

### BACKGROUND

Implantable medical devices, such as electrical stimulation devices, may be used in different therapeutic applications, such as for deep brain stimulation (DBS), spinal cord stimulation (SCS), pelvic stimulation, gastric stimulation, peripheral nerve stimulation, or functional electrical stimulation of a target tissue site within a patient. An electrical stimulation device may be used to treat a variety of symptoms or conditions of a patient, such as chronic pain, tremor, Alzheimer's disease, Parkinson's disease, other types of movement disorders, seizure disorders (e.g., epilepsy), urinary or fecal incontinence, sexual dysfunction, obesity, mood disorders, gastroparesis, or diabetes. In some therapy systems, an implantable electrical stimulator delivers electrical therapy to a target tissue site within a patient with the aid of one or more electrodes, which may be deployed by medical leads. In further embodiments a catheter may be placed by the insertion guide to deliver therapeutic fluids.

### SUMMARY

In general, the disclosure relates to methods, systems, and devices for positioning a device in a body, wherein one system includes a stylet including a proximal end and a distal end, the stylet formed of an elongate cylindrical shape of a uniform diameter, and an insertion guide including a proximal end and a distal end, the insertion guide configured to be inserted into the body in combination with the stylet and being formed of a biodegradable material.

Another embodiment includes a method of inserting a medical device to a desired position in the body, the method including inserting a combination insertion guide and a stylet along a predetermined trajectory and to a predetermined depth in the body, the stylet positioned in a lumen of the insertion guide and providing a stiffness suitable for inserting the combination through the body to the desired location, removing the stylet and leaving the insertion guide in place to provide a pathway for the medical device, and inserting the medical device through the pathway provided by the insertion guide to the desired position, whereby the insertion guide is a tubular structure with a proximal and distal end, the distal end being closed to provide a known stop position in the brain and the proximal end being open for removal of the stylet and insertion of the medical device, the insertion guide formed of biodegradable material.

Another aspect system for providing deep brain stimulation may include a stimulator, a lead, and an electrode includes an insertion guide formed of a sleeve having a lumen therein and extending along the length thereof, the sleeve being tubular in shape and formed of a biodegradable material, and a rod configured to be disposed coaxially within said sleeve and wherein a portion of the length of said rod extends outwardly from the proximal end of said sleeve, the rod for providing a desired stiffness to the combination sleeve and rod for insertion into the brain and configured to be removed from the sleeve after the combination rod and sleeve reaches a desired position in the brain, the sleeve configured to remain in place and provide a path way for the lead to be inserted into the desired position in the brain.

### BRIEF DESCRIPTION OF DRAWINGS

FIG. 1 is a conceptual diagram illustrating an example therapy system that delivers therapy to a patient to manage a disorder of the patient.

FIG. 2 is a functional block diagram illustrating components of an implantable medical device of the therapy system illustrated in FIG. 1.

FIG. 3 is a functional block diagram illustrating components of an example external programmer of the therapy system illustrated in FIG. 1.

FIG. 4 is a perspective view of an insertion guide of the present invention.

FIGS. 5A-E illustrate the insertion of the electrode utilizing the insertion guide of FIG. 4.

### DETAILED DESCRIPTION

The present invention includes an insertion guide configured to be inserted in combination with a stylet wherein the insertion guide is left in the body after the stylet is removed. The insertion guide provides a pathway or conduit for a stimulation lead, a catheter, medical device, or other therapeutic device to a desired location. The insertion guide is further made of biodegradable material such that, after the lead is inserted through the insertion guide, the material forming the insertion guide biodegrades and is absorbed by the body. The below description describes the insertion guide in terms of inserting a lead, but such description should not be interpreted in a limiting sense. The insertion guide may be utilized to insert a lead, a catheter, sensor, monitor or other medical devices to a selected location in the body, including the brain or other areas of the anatomy. The catheter may be utilized as a pathway to deliver a therapeutic fluid to a desired location.

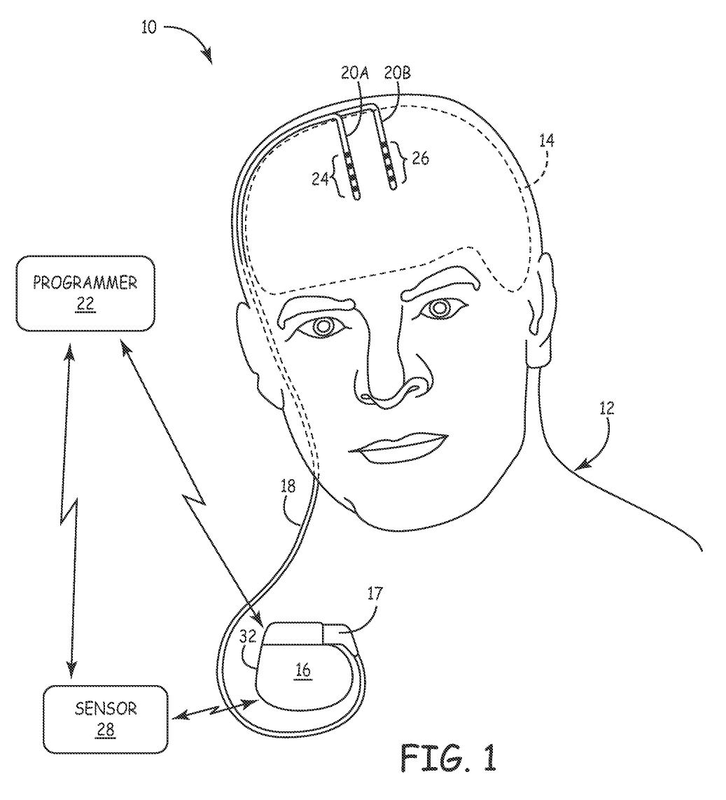

FIG. 1 is a conceptual diagram illustrating an example therapy system 10 that delivers therapy to patient 12 to manage a disorder of patient 12. In some examples, therapy system 10 may deliver therapy to patient 12 to manage a neurological disorder of patient 12. For example, therapy system 10 may provide therapy to manage symptoms of a psychological disorder, a mood disorder, a movement disorder, a cognitive disorder, a sleep disorder, a seizure disorder, or neurodegenerative impairment. In some examples, therapy system 10 may provide therapy to patient 12 to manage Alzheimer's disease. Patient 12 ordinarily will be a human patient. In some cases, however, therapy system 10 may be applied to other mammalian or non-mammalian non-human patients. While examples of the disclosure are described with regard to treatment of a cognitive disorder such as Alzheimer's disease, in other examples, therapy system 10 may provide therapy to manage symptoms of other patient conditions.

Therapy system 10 includes implantable medical device (IMD) 16, lead extension 18, one or more leads 20A and 20B (collectively “leads 20” and generally “lead 20”) with respective sets of electrodes 24, 26, medical device programmer 22, and sensor 28, which may be external to patient 12 or implanted within patient 12. IMD 16 includes a therapy module that includes a stimulation generator that generates and delivers electrical stimulation therapy to one or more regions of brain 14 of patient 12 via the electrodes 24, 26 of leads 20A and 20B, respectively. In the example shown in FIG. 1, therapy system 10 may be referred to as deep brain stimulation (DBS) system because IMD 16 provides electrical stimulation therapy directly to tissue within brain 14, e.g., a tissue site under the dura mater of brain 14. In other examples, leads 20 may be positioned to deliver therapy to a surface of brain 14, e.g., the cortical surface of brain 14.

In the example shown in FIG. 1, IMD 16 may be implanted within a subcutaneous pocket above the clavicle of patient 12. In other examples, IMD 16 may be implanted within other regions of patient 12, such as a subcutaneous pocket in the abdomen or buttocks of patient 12 or proximate the cranium of patient 12. Implanted lead extension 18 is coupled to IMD 16 via a connector block (also referred to as a header), which may include, for example, electrical contacts that electrically couple to respective electrical contacts on lead extension 18. The electrical contacts electrically couple the electrodes 24, 26 carried by leads 20 to IMD 16. Lead extension 18 traverses from the implant site of IMD 16 within a chest cavity of patient 12, along the neck of patient 12 and through the cranium of patient 12 to access brain 14. Generally, IMD 16 is constructed of a biocompatible material that resists corrosion and degradation from bodily fluids. IMD 16 may comprise a hermetic housing 32 that substantially encloses components, such as a processor, therapy module, and memory.

Electrical stimulation may be delivered to one or more regions of brain 14, which may be selected based on many factors, such as the type of patient condition for which therapy system 10 is implemented to manage. In some examples, leads 20 may be implanted within the right and left hemispheres of brain 14 (e.g., as illustrated in FIG. 1) while, in other examples, both of leads 20 may be implanted within one of the right or left hemispheres. Other implant sites for leads 20 and IMD 16 are contemplated. For example, in some examples, IMD 16 may be implanted on or within the cranium. In addition, in some examples, leads 20 may be coupled to a single lead that is implanted within one hemisphere of brain 14 or implanted through both right and left hemispheres of brain 14.

Leads 20 may be positioned to deliver electrical stimulation to one or more target tissue sites within brain 14 to manage patient symptoms associated with a disorder of patient 12. Leads 20 may be implanted to position electrodes 24, 26 at desired locations of brain 14 through respective holes in cranium. Leads 20 may be placed at any location within brain 14 such that electrodes 24, 26 are capable of providing electrical stimulation to target tissue sites within brain 14 during treatment. Different neurological or psychiatric disorders may be associated with activity in one or more regions of brain 14, which may differ between patients. As described in further detail below, in some examples, activity in the cortex and thalamus may be indicative of an Alzheimer's state (e.g., a state in which one or more symptoms of Alzheimer's disease are observed by patient 12, a patient caretaker or a clinician, or a state in which synchronization of a bioelectrical brain signal sensed in the cortex or thalamus is observed).

In the example shown in FIG. 1, electrodes 24, 26 of leads 20 are shown as ring electrodes. Ring electrodes may be relatively easy to program and are typically capable of delivering an electrical field to any tissue adjacent to leads 20 (e.g., in all directions away from an outer perimeter of leads 20). In other examples, electrodes 24, 26 of leads 20 may have different configurations. For example, electrodes 24, 26 of leads 20 may have a complex electrode array geometry that is capable of producing shaped electrical fields. The complex electrode array geometry may include multiple electrodes (e.g., partial ring or segmented electrodes) around the perimeter of each lead 20, rather than a ring electrode. In this manner, electrical stimulation may be directed in a specific direction from leads 20 (e.g., in a direction less than around the entire outer perimeter of leads 20) to enhance therapy efficacy and reduce possible adverse side effects from stimulating a large volume of tissue. In some examples, outer housing 32 of IMD 16 may include one or more stimulation and/or sensing electrodes. For example, housing 32 may comprise an electrically conductive material that is exposed to tissue of patient 12 when IMD 16 is implanted in patient 12, or an electrode can be attached to housing 32. In alternative examples, leads 20 may have shapes other than elongated cylinders as shown in FIG. 1. For example, leads 20 may be paddle leads, spherical leads, bendable leads, or any other type of shape effective in treating patient 12.

In some examples, the location of the electrodes 24, 26 within brain 14 can be determined based on analysis of a bioelectrical brain signal of the patient sensed via one or more of the electrodes 24, 26. For example, a particular physiological structure (e.g., the STN) may exhibit a unique electrical signal and, thus, facilitate positioning of the electrodes of the lead at the desired implant location (e.g., near the target tissue) through monitoring of the bioelectrical brain signal.

In the examples described herein, for treatment of a cognitive disorder (e.g., Alzheimer's disease), leads 20 may be implanted to deliver electrical stimulation to various portions of brain 14 of patient 12, such as the anterior thalamic nucleus, the internal capsule, the cingulate cortex (including the anterior cingulate gyms), the fornix, the mammillary bodies, the mammillothalamic tract (mammillothalamic fasciculus), the hippocampus, the Basal Nucleus of Meynert (NBM), the medial septal nucleus, the thalamic reticular nucleus the orbitofrontal cortex, the locus coeruleus, the raphe nucleus, the substantia nigra, the amygdala, the interior thalamus, the hypothalamus, and other portions of the thalamus and the limbic system. In some examples, leads 20 may be implanted to deliver electrical stimulation to portions of brain 14 that are more posterior than frontal such that electrical stimulation activates a relatively large portion of brain 14.

Although leads 20 are shown in FIG. 1 as being coupled to a common lead extension 18, in other examples, leads 20 may be coupled to IMD 16 via separate lead extensions. In yet other examples, leads 20 may be directly coupled to IMD 16. In addition, although FIG. 1 illustrates system 10 as including two leads 20A and 20B coupled to IMD 16 via lead extension 18, in some examples, system 10 may include one lead or more than two leads.

Leads 20 may deliver electrical stimulation to treat any number of neurological disorders or diseases in addition to cognitive disorders, such as seizure disorders, movement disorders, or psychiatric disorders. Examples of movement disorders include a reduction in muscle control, motion impairment, or other movement problems, such as rigidity, bradykinesia, rhythmic hyperkinesia, nonrhythmic hyperkinesia, dystonia, tremor, and akinesia. Movement disorders may be associated with patient disease states, such as Parkinson's disease or Huntington's disease. Examples of psychiatric disorders include major depressive disorder, bipolar disorder, anxiety disorders, posttraumatic stress disorder, dysthymic disorder, and obsessive compulsive disorder. As described above, examples of the disclosure are primarily described with regard to treating a cognitive disorder (e.g., Alzheimer's disease). Treatment of other patient disorders via delivery of therapy to brain 14 is contemplated, such as, for example, with drugs that treat the above listed disorders in addition to other disorders.

Leads 20 may be implanted within a desired location of brain 14 via any suitable technique, such as through respective burr holes in a skull of patient 12 or through a common burr hole in the cranium. Leads 20 may be placed at any location within brain 14 such that electrodes 24, 26 of leads 20 are capable of providing electrical stimulation to targeted tissue during treatment. Leads 20 may include an internal stylet that provides stiffness during insertion, but that is later removed so that the lead 20 is flexible for long-term comfort and safety. In some embodiments, the lead 20 may be inserted through a cannula (not shown) that guides the lead to a position approximately 18 mm or less from the desired final position. The lead is traversed the final distance through the brain with the internal stylet providing the required stiffness. In other embodiments, a microelectrode recording lead (MER lead) is first placed into the brain through a cannula, which may be the same or different from the cannula/stylet that later guides the electrode. The MER lead may help the clinician find the desired final position for the electrodes 24, 26 of lead 20.

Electrical stimulation generated from the stimulation generator (not shown in FIG. 1) within the therapy module of IMD 16 may help treat (e.g., mitigate symptoms or improve the patient condition) associated with the patient's disorder. For example, in treatment of cognitive disorders such as Alzheimer's disease, electrical stimulation delivered to a target tissue site within brain 14 can help improve basic cognitive functions, e.g., memory processing, perception, problem solving, and language, that may be negatively affected by the cognitive disorder.

The particular parameter values that define the electrical stimulation that activates a neural network in brain 14 of patient 12 in order to treat a cognitive disorder of patient 12 (e.g., the amplitude or magnitude of the stimulation signals, the duration of each signal, the waveform of the stimuli, e.g., rectangular, sinusoidal or ramped signals, the frequency of the signals, and the like) may be specific for the particular target stimulation site (e.g., the portion of brain 14 to which electrical stimulation therapy is delivered). In addition, the particular parameter values may be specific to the particular patient and to the particular patient disorder. In some examples, a processor of therapy system 10 (e.g., a processor of programmer 22 or IMD 16) controls delivery of electrical stimulation by activating electrical stimulation, deactivating electrical stimulation, increasing the intensity of electrical stimulation, or decreasing the intensity of electrical stimulation delivered to brain 14.

Therapy system 10 may also store a plurality of stimulation programs (e.g., a set of electrical stimulation parameter values). Where IMD 16 delivers electrical stimulation in the form of electrical pulses, for example, the stimulation therapy may be characterized by selected pulse parameters, such as pulse amplitude, pulse rate, and pulse width. In addition, if different electrodes are available for delivery of stimulation, the therapy may be further characterized by different electrode combinations, which can include selected electrodes and their respective polarities.

During the trial stage, a plurality of stimulation programs may be tested and evaluated for efficacy. Stimulation programs may be selected for storage within IMD 16 based on the results of the trial stage. Therefore, the trial stage may be useful for customizing the therapy parameter values stored and implemented by IMD 16 for a particular patient 12.

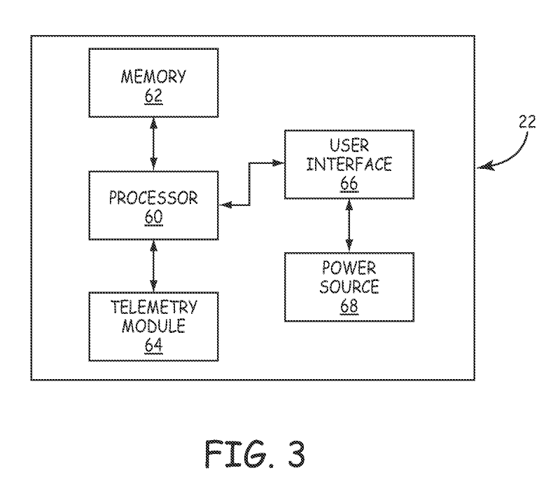

In addition to delivering therapy to manage a disorder of patient 12, therapy system 10 may monitor one or more bioelectrical brain signals of patient 12. For example, IMD 16 may include a sensing module (e.g., sensing module 44 of FIG. 3) that senses bioelectrical brain signals within one or more regions of brain 14. In the example shown in FIG. 1, the signals sensed by electrodes 24, 26 are conducted to the sensing module within IMD 16 via conductors within the respective lead 20A, 20B. In some examples, a processor of IMD 16 or another device (e.g., programmer 22) monitors the bioelectrical signals within brain 14 of patient 12 and controls delivery of electrical stimulation therapy to brain 14 based on the monitored bioelectrical brain signals to provide therapy to patient 12 in a manner that effectively treats a cognitive disorder of patient 12.

In some examples, the sensing module of IMD 16 may receive the bioelectrical signals from electrodes 24, 26 or other electrodes positioned to monitor bioelectrical brain signals of patient 12 (e.g., if housing 32 of IMD 16 is implanted in brain 14, an electrode of housing 32 can be used to sense bioelectrical brain signals and/or deliver stimulation to brain 14). Electrodes 24, 26 may also be used to deliver electrical stimulation from the therapy module to target sites within brain 14 as well as to sense brain signals within brain 14. However, IMD 16 can also use separate sensing electrodes to sense the bioelectrical brain signals. In some examples, the sensing module of IMD 16 may sense bioelectrical brain signals via one or more of the electrodes 24, 26 that are also used to deliver electrical stimulation to brain 14. In other examples, one or more of electrodes 24, 26 may be used to sense bioelectrical brain signals, while one or more different electrodes 24, 26 may be used to deliver electrical stimulation.

Depending on the particular stimulation electrodes and sense electrodes used by IMD 16, IMD 16 may monitor brain signals and deliver electrical stimulation to the same region of brain 14 or to different regions of brain 14. In some examples, the electrodes used to sense bioelectrical brain signals may be located on the same lead used to deliver electrical stimulation while, in other examples, the electrodes used to sense bioelectrical brain signals may be located on a different lead than the electrodes used to deliver electrical stimulation. In some examples, a brain signal of patient 12 may be monitored with external electrodes, e.g., scalp electrodes. Moreover, in some examples, the sensing module that senses bioelectrical brain signals of brain 14 (e.g., the sensing module that generates an electrical signal indicative of the activity within brain 14) may be positioned in a physically separate housing from outer housing 32 of IMD 16. However, in the example shown in FIG. 1 and the example primarily referred to herein for ease of description, the sensing module and therapy module of IMD 16 are enclosed within a common outer housing 32. Other sensing and stimulation electrode configurations than those described above may also be used.

The bioelectrical brain signals monitored by IMD 16 may reflect changes in electrical current produced by the sum of electrical potential differences across brain tissue. Examples of the monitored bioelectrical brain signals include, but are not limited to, an electroencephalogram (EEG) signal, an electrocorticogram (ECoG) signal, a local field potential (LFP) sensed from within one or more regions of brain 14, and/or action potentials from cells within the brain 14. As described in further detail below, therapy system 10 may control delivery of therapy to brain 14 of patient 12 based on the monitored brain signals of patient 12.

Example characteristics of the brain signals of brain 14 can include time domain characteristics (e.g., an amplitude or frequency) or frequency domain characteristics (e.g., an energy level in one or more frequency bands) of the brain signals sensed by IMD 16 within one or more regions of brain 14. For example, the characteristic of the brain signals may include an absolute amplitude value or a root mean square amplitude value. In addition, the amplitude value may comprise an average, peak, mean or instantaneous amplitude value over a period of time or a maximum amplitude or an amplitude in a particular percentile of the maximum (e.g., an amplitude value that represents 95% of the maximum amplitude value).

As another example, the characteristic of the brain signal may include the frequency, amplitude, and phase of the bioelectrical brain signal sensed within one or more regions of brain 14 of patient 12. The frequency, amplitude, and phase of the bioelectrical brain signal may indicate the oscillations in the brain signal. The oscillations in the sensed bioelectrical brain signal may represent the rhythmic or repetitive neural activity in brain 14. The neural oscillations may be determined based on one or more frequency domain characteristics of the bioelectrical brain signal.

In some examples, as illustrated in FIG. 1, therapy system 10 may also include sensor 28. In addition to electrodes 24, 26, sensor 28 can also measure a physiological response of patient 12 that can be indicative of a particular state of brain 14. For example, sensor 28 may be configured to measure physiological parameters such as galvanic skin response, respiratory rate, heart rate, body temperature, and/or muscle activity of patient 12, and transmit the measurements to IMD 16 or another component of therapy system 10 to determine whether brain 14 is in a particular state. Other physiological responses are also contemplated. As discussed above, in some examples, sensor 28 may be external to patient 12 and may communicate with IMD 16 and/or programmer 22 via a wireless communication link. In other examples, sensor 28 may be implanted within patient 12 and may communicate with IMD 16 via a wired or wireless communication link, and communicate with programmer 22 via a wireless communication link. In examples in which sensor 28 is implanted in patient 14, sensor 28 may be physically separate from IMD 16 or may be incorporated in IMD 16.

External programmer 22 wirelessly communicates with IMD 16 as needed to provide or retrieve therapy information. Programmer 22 is an external computing device that the user, e.g., the clinician and/or patient 12 or patient caretaker, may use to communicate with IMD 16. For example, programmer 22 may be a clinician programmer that the clinician uses to communicate with IMD 16 and program one or more therapy programs for IMD 16. Additionally or alternatively, programmer 22 may be a patient programmer that allows patient 12 to select programs and/or view and modify therapy parameters. The clinician programmer may include more programming features than the patient programmer includes. In other words, more complex or sensitive tasks may only be allowed by the clinician programmer to prevent an untrained patient from making undesirable changes to IMD 16.

Programmer 22 may be a hand-held computing device with a display viewable by the user and an interface for providing input to programmer 22 (i.e., a user input mechanism). For example, programmer 22 may include a small display screen (e.g., a liquid crystal display (LCD) or a light emitting diode (LED) display) that presents information to the user. In addition, programmer 22 may include a touch screen display, keypad, buttons, a peripheral pointing device or another input mechanism that allows the user to navigate though the user interface of programmer 22 and provide input. If programmer 22 includes buttons and a keypad, the buttons may be dedicated to performing a certain function, i.e., a power button, or the buttons and the keypad may be soft keys that change in function depending upon the section of the user interface currently viewed by the user. Alternatively, the screen (not shown) of programmer 22 may be a touch screen that allows the user to provide input directly to the user interface shown on the display. The user may use a stylus or their finger to provide input to the display.

In other examples, programmer 22 may be a larger workstation or a separate application within another multi-function device, rather than a dedicated computing device. For example, the multi-function device may be a notebook computer, tablet computer, workstation, cellular phone, personal digital assistant or another computing device that may run an application that enables the computing device to operate as a secure medical device programmer 22. A wireless adapter coupled to the computing device may enable secure communication between the computing device and IMD 16.

When programmer 22 is configured for use by the clinician, programmer 22 may be used to transmit initial programming information to IMD 16. This initial information may include hardware information, such as the type of leads 20, the arrangement of electrodes 24, 26 on leads 20, the position of leads 20 within brain 14, initial programs defining therapy parameter values, and any other information that may be useful for programming into IMD 16. Programmer 22 may also be capable of completing functional tests (e.g., measuring the impedance of electrodes 24, 26 of leads 20).

The clinician may also store therapy programs within IMD 16 with the aid of programmer 22. During a programming session, the clinician may determine one or more stimulation programs that may effectively induce a desired state in brain 14 of patient 12. For example, the clinician may select one or more electrode combinations with which stimulation is delivered to brain 14 to generate the desired state. During the programming session, the clinician may evaluate the efficacy of the one or more electrode combinations based on one or more physiological parameters of patient 12 (e.g., heart rate, respiratory rate, galvanic skin response, bioelectrical brain signals, etc.). In some examples, programmer 22 may assist the clinician in the creation/identification of stimulation programs by providing a methodical system for identifying potentially beneficial stimulation parameter values. In some examples, the processor of programmer 22 may calculate and display one or more therapy metrics for evaluating and comparing therapy programs available to delivery of therapy from IMD 16 to patient.

The clinician may also program one or more physiological parameters with which IMD 16 may use to detect certain brain states of patient 12 used in controlling therapy delivery or monitoring patient 12. For example, the clinician may select one or more signal characteristics (e.g., a time domain or frequency domain characteristic) that indicate a portion of brain 28 associated with one or more symptoms of Alzheimer's disease.

Programmer 22 may also be configured for use by patient 12. When configured as a patient programmer, programmer 22 may have limited functionality (compared to a clinician programmer) in order to prevent patient 12 from altering critical functions of IMD 16 or applications that may be detrimental to patient 12. In this manner, programmer 22 may only allow patient 12 to adjust values for certain therapy parameters or set an available range of values for a particular therapy parameter.

Programmer 22 may also provide an indication to patient 12 when therapy is being delivered, when patient input has triggered a change in therapy or when the power source within programmer 22 or IMD 16 needs to be replaced or recharged. For example, programmer 22 may include an alert LED, may flash a message to patient 12 via a programmer display, generate an audible sound or somatosensory cue to confirm patient input was received, e.g., to indicate a patient state or to manually modify a stimulation parameter.

Whether programmer 22 is configured for clinician or patient use, programmer 22 is configured to communicate with IMD 16 and, optionally, another computing device, via wireless communication. Programmer 22, for example, may communicate via wireless communication with IMD 16 using radio frequency (RF) telemetry techniques known in the art. Programmer 22 may also communicate with another programmer or computing device via a wired or wireless connection using any of a variety of local wireless communication techniques, such as RF communication according to the 802.11 or Bluetooth specification sets, infrared (IR) communication according to the IRDA specification set, or other standard or proprietary telemetry protocols. Programmer 22 may also communicate with other programming or computing devices via exchange of removable media, such as magnetic or optical disks, memory cards or memory sticks. Further, programmer 22 may communicate with IMD 16 and another programmer via remote telemetry techniques known in the art, communicating via a local area network (LAN), wide area network (WAN), public switched telephone network (PSTN), or cellular telephone network, for example.

Therapy system 10 may be implemented to provide chronic stimulation therapy to patient 12 over the course of several months or years. However, system 10 may also be employed on a trial basis to evaluate therapy before committing to full implantation. If implemented temporarily, some components of system 10 may not be implanted within patient 12. For example, patient 12 may be fitted with an external medical device, such as a trial stimulator, rather than IMD 16. The external medical device may be coupled to percutaneous leads or to implanted leads via a percutaneous extension. If the trial stimulator indicates DBS system 10 provides effective treatment to patient 12, the clinician may implant a chronic stimulator within patient 12 for relatively long-term treatment.

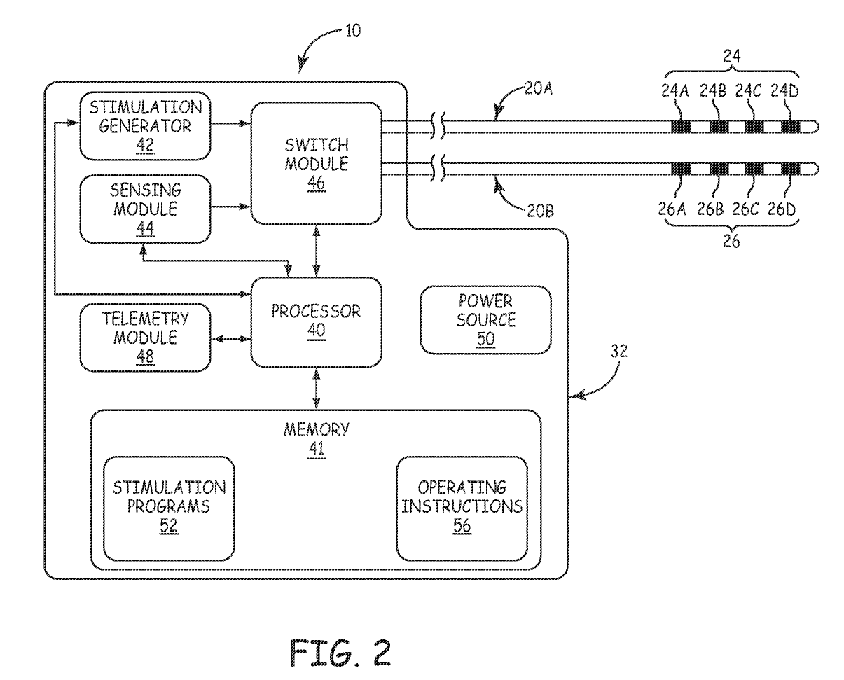

FIG. 2 is a functional block diagram illustrating components of IMD 16. In the example shown in FIG. 2, IMD 16 includes processor 40, memory 41, stimulation generator 42, sensing module 44, switch module 46, telemetry module 48, and power source 50. Memory 41 may include any volatile or non-volatile media, such as a random access memory (RAM), read only memory (ROM), non-volatile RAM (NVRAM), electrically erasable programmable ROM (EEPROM), flash memory, and the like. Memory 41 may store computer-readable instructions that, when executed by processor 40, cause IMD 16 to perform various functions described herein.

In the example shown in FIG. 2, memory 41 stores stimulation programs 52 and operating instructions 56 in separate memories within memory 41, or in separate modules within memory 41. Each stored stimulation program 52 (which can also be referred to as a type of therapy program) defines a particular set of electrical stimulation parameters, e.g., a stimulation electrode combination, electrode polarity, frequency, current or voltage amplitude. In examples in which stimulation generator 42 generates and delivers stimulation pulses, the stimulation programs 52 may define values for pulse width and pulse rate of the stimulation signal. In some examples, one or more of the stimulation programs 52 may be stored as a therapy group, e.g., a group of related stimulation programs. The stimulation signals defined by the stimulation programs of the therapy group may be delivered together on an overlapping or non-overlapping (e.g., time-interleaved) basis. In addition, memory 41 may store information related to a schedule according to which electrical stimulation is delivered to brain 14 (e.g., a schedule defining a total amount of time daily that stimulation is delivered to brain 14, a schedule defining particular points in time during which stimulation is delivered, a schedule defining a cyclical basis on which stimulation is delivered, and the like).

Stimulation generator 42, under the control of processor 40, generates stimulation signals for delivery to patient 12 via selected combinations of electrodes 24, 26. Processor 40 controls stimulation generator 42 according to stimulation programs 52 stored in memory 41 to apply particular stimulation parameter values specified by one or more programs, such as amplitude, pulse width, and pulse rate. In some examples, stimulation generator 42 generates and delivers stimulation signals to one or more target portions of brain 14, e.g., Basal Nucleus of Meynert, anterior cingulate gyms, ascending reticular activation system, via a select combination of electrodes 24, 26, where the stimulation signals have a frequency in a range of about 50 Hertz (Hz) to about 250 Hz, a voltage of about 0.1 volts to about 10.5 volts, and a pulse width of about 60 microseconds to about 450 microseconds. In some examples, the stimulation signals have a frequency of 130 Hz, a voltage of about 2 volts, and a pulse width of about 60 microseconds. In addition, in some examples, the stimulation signals have a frequency of 145 Hz, a voltage of about 5 volts, and a pulse width of about 145 microseconds.

Other stimulation parameter values may also be used and may vary depending on the patient and the patient's response to the stimulation. For example, some patients may require higher intensity (e.g., a function of a plurality of stimulation parameter values, such as the frequency, amplitude, and pulse width) stimulation. As another example, depending on the structure of brain 14 that is being activated by the stimulation, lower frequency stimulation may be desirable, such as a frequency of about 50 Hz or less, in order to activate certain structures.

Various target tissue sites within brain 14, stimulation parameter values, and other therapy delivery schedules are contemplated. In some examples, other ranges of stimulation parameter values may also be useful, and may be determined based on the target stimulation site within patient 12, which may or may not be within brain 14. While stimulation pulses are described, stimulation signals may be of any form, such as continuous-time signals (e.g., sine waves) or the like.

In each of the examples described herein, if stimulation generator 42 shifts the delivery of stimulation energy between two stimulation programs and/or two different electrode combinations, processor 40 of IMD 16 may provide instructions that cause stimulation generator 42 to time-interleave stimulation energy between the electrode combinations of the two therapy programs, as described in commonly-assigned U.S. Pat. No. 7,519,431, entitled, “SHIFTING BETWEEN ELECTRODE COMBINATIONS IN ELECTRICAL STIMULATION DEVICE,” which issued on Apr. 14, 2009, the entire content of which is incorporated herein by reference. In the time-interleaved shifting example, the amplitudes of the stimulation signals delivered via the electrode combinations of the first and second therapy program are ramped downward and upward, respectively, in incremental steps until the amplitude of the second electrode combination reaches a target amplitude. The incremental steps may be different between ramping downward or ramping upward. The incremental steps in amplitude can be of a fixed size or may vary, e.g., according to an exponential, logarithmic or other algorithmic change. When the second electrode combination reaches its target amplitude, or possibly before, the first electrode combination can be shut off Other techniques for shifting the delivery of stimulation signals between two therapy programs and/or electrode combinations may be used in other examples.

Processor 40 may include any one or more of a microprocessor, a controller, a digital signal processor (DSP), an application specific integrated circuit (ASIC), a field-programmable gate array (FPGA), discrete logic circuitry, and the functions attributed to processor 40, as well as processors described herein, may be embodied as firmware, hardware, software or any combination thereof.

In the example shown in FIG. 2, the set of electrodes 24 of lead 20A includes electrodes 24A, 24B, 24C, and 24D, and the set of electrodes 26 of lead 20B includes electrodes 26A, 26B, 26C, and 26D. Processor 40 may control switch module 46 to apply the stimulation signals generated by stimulation generator 42 to selected combinations of electrodes 24, 26. In particular, switch module 46 may couple stimulation signals to selected conductors within leads 20, which, in turn, deliver the stimulation signals across selected electrodes 24, 26. Switch module 46 may be a switch array, switch matrix, multiplexer, or any other type of switching module configured to selectively couple stimulation energy to selected electrodes 24, 26 and to selectively sense bioelectrical brain signals with selected electrodes 24, 26. Hence, stimulation generator 42 is coupled to electrodes 24, 26 via switch module 46 and conductors within leads 20. In some examples, however, IMD 16 does not include switch module 46.

Stimulation generator 42 may be a single channel or multi-channel stimulation generator. In particular, stimulation generator 42 may be capable of delivering a single stimulation pulse, multiple stimulation pulses, or a continuous signal at a given time via a single electrode combination or multiple stimulation pulses at a given time via multiple electrode combinations. In some examples, however, stimulation generator 42 and switch module 46 may be configured to deliver multiple channels on a time-interleaved basis. For example, switch module 46 may serve to time divide the output of stimulation generator 42 across different electrode combinations at different times to deliver multiple programs or channels of stimulation energy to patient 12.

Sensing module 44 is configured to sense bioelectrical brain signals of patient 12 via a selected subset of electrodes 24, 26, or with one or more electrodes 24, 26 and at least a portion of a conductive outer housing 32 of IMD 16, an electrode on an outer housing of IMD 16, or another reference. Processor 40 may control switch module 46 to electrically connect sensing module 44 to selected electrodes 24, 26. In this way, sensing module 44 may selectively sense bioelectrical brain signals with different combinations of electrodes 24, 26 (and/or a reference other than an electrode 24, 26).

Although sensing module 44 is incorporated into a common housing 32 with stimulation generator 42 and processor 40 in FIG. 3, in other examples, sensing module 44 is in a physically separate outer housing from outer housing 32 of IMD 16 and communicates with processor 40 via wired or wireless communication techniques.

Telemetry module 48 supports wireless communication between IMD 16 and an external programmer 22 or another computing device under the control of processor 40. Processor 40 of IMD 16 may receive, as updates to stimulation programs, values for various stimulation parameters such as amplitude and electrode combination, via telemetry module 48. The updates to the stimulation programs may be stored within stimulation programs 52 of memory 41. Telemetry module 48 in IMD 16, as well as telemetry modules in other devices and systems described herein, such as programmer 22, may accomplish communication by RF communication techniques. In addition, telemetry module 48 may communicate with external medical device programmer 22 via proximal inductive interaction of IMD 16 with programmer 22. Accordingly, telemetry module 48 may send information to external programmer 22 on a continuous basis, at periodic intervals, or upon request from IMD 16 or programmer 22.

Power source 50 delivers operating power to various components of IMD 16. Power source 50 may include a small rechargeable or non-rechargeable battery and a power generation circuit to produce the operating power. Recharging may be accomplished through proximal inductive interaction between an external charger and an inductive charging coil within IMD 16. In some examples, power requirements may be small enough to allow IMD 16 to utilize patient motion and implement a kinetic energy-scavenging device to trickle charge a rechargeable battery. In other examples, traditional batteries may be used for a limited period.

FIG. 3 is a functional block diagram illustrating components of an example medical device programmer 22 (FIG. 1). Programmer 22 includes processor 60, memory 62, telemetry module 64, user interface 66, and power source 68. Processor 60 controls user interface 66 and telemetry module 64, and stores and retrieves information and instructions to and from memory 62. Programmer 22 may be configured for use as a clinician programmer or a patient programmer. Processor 60 may comprise any combination of one or more processors including one or more microprocessors, DSPs, ASICs, FPGAs, or other equivalent integrated or discrete logic circuitry. Accordingly, processor 60 may include any suitable structure, whether in hardware, software, firmware, or any combination thereof, to perform the functions ascribed herein to processor 60.

A user, such as a clinician or patient 12, may interact with programmer 22 through user interface 66. For example, a clinician may provide input via user interface 66 related to stimulation parameters that define effective stimulation and programmer 22 may transmit the stimulation parameters to IMD 16. As another example, a clinician or another user may provide user input via user interface 66.

User interface 66 includes a display (not shown), such as a LCD or LED display or other type of screen, to present information related to the therapy, such as information related to bioelectrical brain signals sensed via a plurality of sense electrode combinations. In addition, user interface 66 may include an input mechanism to receive input from the user. The input mechanisms may include, for example, buttons, a keypad (e.g., an alphanumeric keypad), a peripheral pointing device or another input mechanism that allows the user to navigate though user interfaces presented by processor 60 of programmer 22 and provide input.

If user interface 66 includes buttons and a keypad, the buttons may be dedicated to performing a certain function, i.e., a power button, or the buttons and the keypad may be soft keys that change function depending upon the section of the user interface currently viewed by the user. Alternatively, the display screen (not shown) of programmer 22 may be a touch screen that allows the user to provide input directly to the user interface shown on the display. The user may use a stylus or their finger to provide input to the display. In other examples, user interface 66 also includes audio circuitry for providing audible instructions or sounds to patient 12 and/or receiving voice commands from patient 12, which may be useful if patient 12 has limited motor functions. Patient 12, a clinician or another user may also interact with programmer 22 to manually select stimulation programs, generate new stimulation programs, modify stimulation programs through individual or global adjustments, and transmit the new programs to IMD 16.

In some examples, at least some of the control of electrical stimulation delivery by IMD 16 may be implemented by processor 60 of programmer 22. For example, processor 60 may perform any of the techniques described herein with respect to processor 40 of IMD 16. For example, in some examples, processor 60 may receive sensed brain signal information from IMD 16 or from a sensing module that is separate from IMD 16. The separate sensing module may, but need not be, implanted within patient 12. Brain signal information may include, for example, a time domain characteristic (e.g., an amplitude) or a frequency domain characteristic (e.g., an energy level in one or more frequency bands) of bioelectrical brain signals monitored by sensing module 44 using one or more of electrodes 24, 26 (FIG. 2). Based on the monitored brain signal information, processor 60 may determine a state of brain 14 and control delivery of electrical stimulation from IMD 16 to patient 12 based on the determined state.

Memory 62 may include instructions for operating user interface 66 and telemetry module 64, and for managing power source 68. Memory 62 may also store any therapy data retrieved from IMD 16 during the course of therapy, stimulation programs, and information related to schedules according to which stimulation can be delivered to brain 14. The clinician may use the therapy data to determine stimulation parameters and treatment plans that can most effectively treat the cognitive disorder of patient 12. Memory 62 may include any volatile or nonvolatile memory, such as RAM, ROM, EEPROM or flash memory. Memory 62 may also include a removable memory portion that may be used to provide memory updates or increases in memory capacities. A removable memory may also allow sensitive patient data to be removed before programmer 22 is used by a different patient.

Wireless telemetry in programmer 22 may be accomplished by RF communication or proximal inductive interaction of external programmer 22 with IMD 16. This wireless communication is possible using telemetry module 64. Accordingly, telemetry module 64 may be similar to the telemetry module contained within IMD 16. In alternative examples, programmer 22 may be capable of infrared communication or direct communication through a wired connection. In this manner, other external devices may be capable of communicating with programmer 22 without needing to establish a secure wireless connection.

Power source 68 delivers operating power to the components of programmer 22. Power source 68 may include a battery and a power generation circuit to produce the operating power. In some examples, the battery may be rechargeable to allow extended operation. Recharging may be accomplished by electrically coupling power source 68 to a cradle or plug that is connected to an alternating current (AC) outlet. In addition, recharging may be accomplished through proximal inductive interaction between an external charger and an inductive charging coil within programmer 22. In other examples, traditional batteries (e.g., nickel cadmium or lithium ion batteries) may be used. In addition, programmer 22 may be directly coupled to an alternating current outlet to operate. Power source 68 may include circuitry to monitor power remaining within a battery. In this manner, user interface 66 may provide a current battery level indicator or low battery level indicator when the battery needs to be replaced or recharged. In some cases, power source 68 may be capable of estimating the remaining time of operation using the current battery.

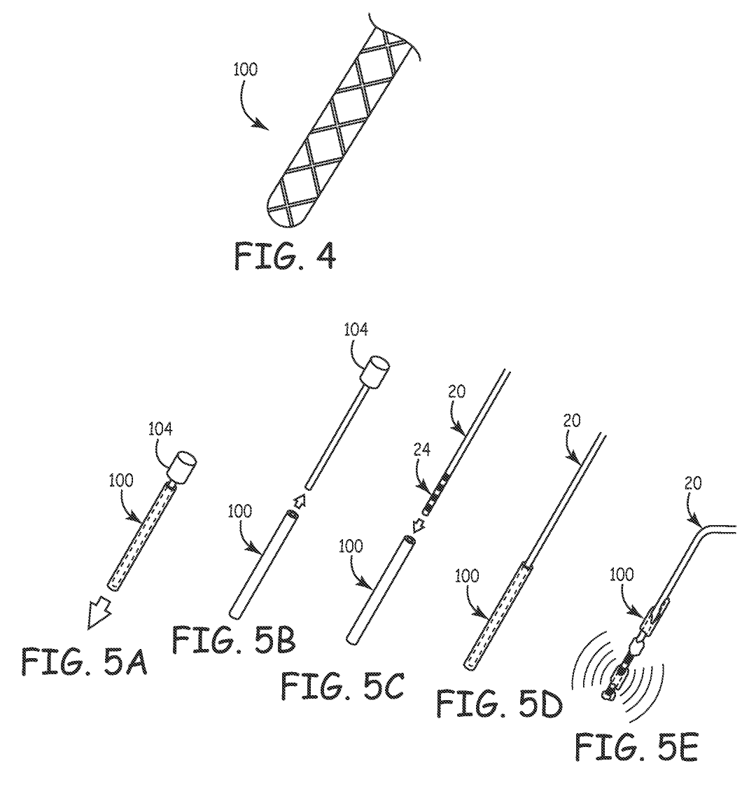

FIG. 4 illustrates one embodiment of the present invention insertion guide 100. The insertion guide 100 may be utilized to aid insertion of devices into the body such as deep brain stimulation leads 20. Leads 20 require some guidance to follow an intended pathway to the targeted tissue and the insertion guide 100 provides a path or conduit formed of biodegradable material. In one embodiment, the insertion guide 100 is formed of a biodegradable polymer formed into a loosely woven mesh. In another embodiment the insertion guide 100 may be a relatively tightly woven mesh. In still further embodiments, the insertion guide 100 may be formed in a variety of ways into the desired cylindrical tube or sock shape. The walls of the insertion guide 20 may be porous or relatively impermeable to liquid. Further, the walls may be relatively thin or thick depending on the desired stiffness, strength, cross section, etc. of the insertion guide 20. As may be further appreciated, the overall width and length of the insertion guide 100 may be different depending on the width and length of the lead 20 to be inserted. As further discussed below, in one or more embodiments, the insertion guide 20 may be shortened by the clinician during implantation by cutting, snipping, or otherwise shortening the insertion guide 100. As is further discussed herein, the instrument guide 100 may also be used to guide other medical instruments, such as, for example, catheters.

The insertion guide 100 of the present embodiment is formed of a biodegradable polymer, such as synthetic or natural bioabsorbable polymers. The biodegradable polymer material may be formed into a generally cylindrical sheath of an appropriate size to fit over the lead 20. As may be appreciated, the polymer that forms the insertion guide 100 may take on a variety of shapes and forms, including woven, braid, coil, mesh, solid, electrospun, and combinations thereof. Electrostatic spinning represents an attractive approach for polymer processing with the opportunity for control over morphology, porosity and composition, and hence mechanical properties, using simple equipment. In electrostatic spinning, polymer solutions are deposited as fibrous mats, with advantage taken of chain entanglements at sufficiently high polymer concentrations in solution to produce continuous fibers.

In one embodiment the insertion guide 100 material may be interwoven strands of the selected biocompatible biodegradable polymer. In further embodiments the insertion guide 100 material may be a formed of a substantially solid sheet of material that is formed into the desired cylindrical shape. In various embodiments, the insertion guide 100 may be relatively stiff or relatively pliable, depending on the desired parameters relating to length, width, stiffness etc. A pliable insertion guide 100 may be somewhat collapsible such that the path created is present but of minimal size until the lead 20 is inserted there through. In further embodiments, the insertion guide 20 may create a sort of tunnel through which the lead 20 is inserted whereby the lead 20 is only required to provide minimal stiffness.

The material that forms the insertion guide 100 may be natural or synthetic. Example synthetic bioabsorbable polymeric materials that can be used include poly(L-lactic acid), polycaprolactone, poly(lactide-co-glycolide), poly(ethylene-vinyl acetate), poly(hydroxybutyrate-covalerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) such as PEO/PLA, polyalkylene oxalates, polyphosphazenes, and polyarylates including tyrosine-derived polyarylates.

Other biodegradable polymers may include natural polymers and polymers derived thereof such as albumin, alginate, casein, chitin, chitosan, collagen, dextran, elastin, proteoglycans, gelatin and other hydrophilic proteins, glutin, zein and other prolamines and hydrophobic proteins, starch and other polysaccharides including cellulose and derivatives thereof (e.g. methyl cellulose, ethyl cellulose, hydroxypropyl cellulose, hydroxy-propyl methyl cellulose, hydroxybutyl methyl cellulose, carboxymethyl cellulose, cellulose acetate, cellulose propionate, cellulose acetate butyrate, cellulose acetate phthalate, cellulose acetate succinate, hydroxypropylmethylcellulose phthalate, cellulose triacetate, cellulose sulphate), poly-1-lysine, polyethylenimine, poly(allyl amine), polyhyaluronic acids, and combinations, copolymers, mixtures and chemical derivatives thereof (substitutions, additions of chemical groups, for example, alkyl, alkylene, hydroxylations, oxidations, and other modifications routinely made by those skilled in the art). Some of these materials may degrade either by enzymatic hydrolysis or exposure to water in vivo, by surface or bulk erosion.

According to another exemplary embodiment, the polymeric materials can be natural bioabsorbable polymers such as, but not limited to, fibrin, fibrinogen, cellulose, starch, collagen, and hyaluronic acid. “Biodegradable”, “bioerodable”, and “bioabsorbable” are used herein interchangeably. In addition, the insertion guide 100 may be formed of two or more polymers that are interwoven, layered, or otherwise physically or mechanically combined to achieve some desired property and shape.

The rate of degradation of the biodegradable material is determined by factors such as configurational structure, copolymer ratio, crystallinity, molecular weight, morphology, stresses, amount of residual monomer, porosity and site of implantation. The insertion guide 100 may biodegrade over a selected and desired period of time, such as a few minutes, one or more hours, 1 or more days, 1-2 weeks, 3 weeks, 3-5 weeks, or more, depending on the selected material and the manner in which the insertion guide 100 is formed. The skilled person will be able to choose the combination of factors and characteristics to optimize the rate of degradation.

In a different embodiment, the materials used for the insertion guide may be incorporated with bioactive agents for release as the biodegradable material gradually undergoes degradation and absorption into the body. Bioactive agents shall include any compound that engenders a physiological, biological or therapeutic effect in the host. Exemplary, non-limiting examples of bioactive agents include pharmacological and biological (small molecules, anti-sense nucleotides, peptides, proteins, hormones, DNA or RNA fragments, genes, antibodies, etc.) entities, and various combinations thereof. In general, depending on the nature of the released agent, some non-limiting examples of the biological effects observed may be decreased inflammation, pro-healing responses (such as boosting cell growth, improving cell morphology, inhibiting cell degeneration), reduction in plaque formation (for Alzheimer's), enhanced neuroprotective effects, cancer suppressing agents, and reversal of neurodegeneration.

The stylet 104 may be any type of stylet known to those of skill in the art, and may be a rod a cannula or other similar articles for adding stiffness to the insertion guide 100 for placement into the desired location through the desired path. The stylet 104 is generally cylindrical and formed of a stiff biocompatible metal or metallic alloy to temporarily provide the insertion guide 100 sufficient column strength to support tunneling. Example materials for the stylet may include stainless steel, nickel, tungsten, alloys of the same, and ceramics and other materials known to those in the art. They stylet may be fully or partially coated with ethylene tetrafluoroethylene, polytetrafluoroethylene, polyimide, polyamide or parylene material and/or treated with plasma or other surface treatments for electrical insulation or to reduce friction. The stylet 104 may be formed of a desired material and to a desired thickness and length to achieve placement of the insertion guide 20 desired location. In addition, in embodiments without a straight trajectory path or conduit, the stylet 104 may be curved or may be malleable to form a desired shape.

Placement of the lead 20 utilizing the insertion guide 100 will be herein described with reference to FIGS. 5A-E. In order to provide the guidance for the lead 20, a stylet 104 is first inserted into the insertion guide 100 and the combination is guided to the desired target in the brain. Placement of the insertion guide 100 and stylet 104 may be accomplished through several methods known to those in the art, including using various image registration techniques, planning software tools, and head frames or other guidance equipment. In one embodiment, the insertion guide 100 is placed through a burr hole in the skull. In addition, microelectrode recording may or may not be first conducted to determine a desired depth. Once the combination insertion guide 100 and stylet 104 reaches the desired depth, the stylet 104 is removed. Before or after the stylet 104 is removed, the insertion guide 100 may be trimmed to extend a desired distance from the patient.

When the stylet 104 is removed, the insertion guide 100 is left behind, providing a path to the targeted tissue. The lead 20 is then inserted through the insertion guide 100 to the desired target. The lead 20 may or may not include an internal stylet to provide stiffness for traveling along the path formed by the insertion guide 100. In other embodiments, the internal stylet may be relatively thin. Once the lead 20 is placed in the desired location, over time the insertion guide 100 biodegrades and is absorbed into the body. As may be appreciated, it is desirable that the insertion guide 100 biodegrades at a known rate or during a known period. Stimulation may commence immediately depending on the materials that were utilized to form the insertion guide 100 and the thickness of the same. In further embodiments, stimulation and programming may be delayed until the insertion guide 100 has completely or substantially degraded. In one embodiment, the distal end of the insertion guide 100 is shaped like the end of a sock and functions as a depth stop, while the proximal end remains open for the introduction of the lead 20. In other embodiments the insertion guide 100 may include an open end or an opening somewhere along the length. An open end or opening along the length may be useful for infusion of therapeutic fluids.

The lead 20 may be any electrical stimulation lead known to those of skill in the art. The lead may include one or more electrodes for stimulation of the desired region. In addition, the lead may be of several diameters, including 0.7 mm to 1.0 mm, 1.0 mm to 1.3 mm, about 1.3 mm, and smaller or larger. As previously discussed, the lead 20 may be coupled to an internal stylet 104 that is removed after the lead 20 is positioned. In other embodiments, the stylet 104 may be excluded all together.

As previously discussed, in certain situations the lead 20 is extended from the insertion guide 100 some distance to the target. In such situations, the insertion guide 100 may allow the lead 20 to be extended from the distal end to reach the target. As may be appreciated, the insertion guide 100 must be mated to the stylet 104 such that the insertion guide 100 can have an open end but is not pushed back along the length of the stylet 104 during insertion due to greater friction caused by contact with brain tissue. The distal end of the insertion guide 100 may be a fixed diameter opening to allow the lead 20 to be pushed further into the brain tissue. In still further embodiments, the end of the insertion guide 100 may be closed but operable to expand, dilate, or otherwise mechanically open to allow for the lead 20 to be extended from the insertion guide 100. As may be appreciated, a variety of woven, interleaved, or other structural features may be utilized to affect such a feature. In further embodiments, the insertion guide 100 may be useful if placing a lead 20 along a non-straight or curved trajectory, as the polymer may be axially flexible along the chosen path.

The combination of the insertion guide 100 and the stylet 104 may be narrow to reduce any possible damage of tissue at the targeted stimulation site. In addition, the material that forms the insertion guide 100 may be relatively thin to reduce the distance between the electrodes 24, 26 on the lead and the targeted tissue. As may be appreciated, the distance between the electrodes 24, 26 and the targeted tissue will be reduced as the insertion guide 100 biodegrades. In further embodiments, the insertion guide 100 may have a thinner outer wall on a distal end of the insertion guide 100 as compared to those areas proximal to where the electrodes 24, 26 of the lead 20 will ultimately rest. In one embodiment utilizing a closed end insertion guide 100, the internal stylet may be excluded from the electrode 20 due to the insertion guide 100 providing guidance all the way to the targeted tissue. In further embodiments, thinner insertion guide 100 and electrode 20 combinations may enable multiple electrodes 20 may be inserted using multiple biodegradable insertion guides 100 to provide more stimulation volume without damaging as much tissue as with previous leads 20.

The insertion guide 100 may be referred to by other names such as a cannula, sock, tube, sleeve, etc. without changing the nature and scope of the invention. In those cases where the insertion guide 100 is utilized to place a catheter, the insertion guide 100 may include an open end or a mesh that allows fluid to pass without interruption.

The techniques described in this disclosure, including those attributed to programmer 22, IMD 16, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

In one example, solutions of polycaprolactone (PCL) in chloroform were used to form the insertion guide 100. PCL was chosen because it is a well-known biocompatible and biodegradable material. An electrospinning set-up consisted of a nozzle (Terronics), a ground electrode mounted onto a stylet, and a high voltage supply. The solutions were delivered via a syringe pump, and as the electrical potential was applied, a jet was created. The resulting fibers were collected on the rotating stylet to produce a sheet (‘sock’) of non-woven fabric. Typical fiber thickness was in the 15-10 μm range. This technique resulted in sheets with good mechanical properties, and a scaffold that due to its elastic properties stayed on the stylet (like a sock).

In further embodiments the present invention may be utilized for guiding a drug delivery catheter or other therapy delivery device to a desired location. The catheter may be placed down the insertion guide 100 after the stylet 104 is removed so that infusion of a therapeutic fluid can be achieved. The therapeutic fluid may contain drugs or other materials to be administered. In still other embodiments, other medical devices such as sensors, may also be implanted with the present invention.

In still further embodiments a location device may be integrated into the insertion guide 100 or stylet 104 for verification of placement location. A coil or other location device may be located in the tip or near the tip to determine the final location of the insertion guide 100.

Such hardware, software, firmware may be implemented within the same device or within separate devices to support the various operations and functions described in this disclosure. While the techniques described herein are primarily described as being performed by processor 40 of IMD 16 and/or processor 60 of programmer 22, any one or more parts of the techniques described herein may be implemented by a processor of one of IMD 16, programmer 22, or another computing device, alone or in combination with each other.

In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed to support one or more aspects of the functionality described in this disclosure.

Various examples have been described. These and other examples are within the scope of the following claims.

1. A system for positioning a device in a body, the system comprising:
a stylet including a proximal end and a distal end, the stylet formed of an elongate cylindrical shape of a uniform diameter; and
an insertion guide including a proximal end and a distal end, the insertion guide configured to be inserted into the body in combination with the stylet and being formed of a biodegradable material.

2. The system of claim 1 wherein the combination stylet and insertion guide is inserted into the body along a desired surgical path.

3. The system of claim 1 whereby the system is configured such that, after the stylet and insertion guide are inserted into the body, the stylet is removed and the insertion guide is left in the body to form a path for positioning the device in the body.

4. The system of claim 3 wherein the path is not straight.

5. The system of claim 1 wherein the insertion guide is formed of a tubular shape with a lumen there through to receive the stylet during insertion into the body and the device after the stylet is removed.

6. The system of claim 1 wherein the insertion guide is formed of a material that is one or more of woven, braid, coil, mesh, solid, electrospun, and combinations thereof.

7. The system of claim 1 whereby the insertion guide degrades at a known rate

8. The system of claim 1 whereby the insertion guide is formed of a material that is one or more of synthetic or natural polymer.

9. The system of claim 1 whereby the device positioned in the body is one or more of a lead, electrode, catheter, or sensor.

10. The system of claim 1 whereby the insertion guide has a closed distal end to provide a known stop position for the device when the device is inserted into the body.

11. The system of claim 1 whereby the insertion guide has an open distal end through which the device can be extended.

12. A method of inserting a medical device to a desired location in the body, the method comprising
inserting a combination insertion guide and a stylet along a predetermined trajectory and to a predetermined depth in the body, the stylet positioned in a lumen of the insertion guide and providing a stiffness suitable for inserting the combination through the body to the desired location;
removing the stylet and leaving the insertion guide in place to provide a pathway for placement of a medical device; and
inserting the medical device through the pathway provided by the insertion guide to the desired location, whereby the insertion guide is a tubular structure with a proximal and distal end, the distal end being closed to provide a known stop position in the brain and the proximal end being open for removal of the stylet and insertion of the medical device, the insertion guide formed of biodegradable material.

13. The method of claim 12 wherein the desired location in the body further comprises the brain.

14. The method of claim 12 wherein the medical device further comprises one of a lead, electrode, sensor or a catheter.

15. The method of claim 12 further comprising following a predetermined path when inserting the combination insertion guide and stylet, the predetermined path being straight, non-straight or curved.

16. The method of claim 12 wherein the wall of the insertion guide is formed of one or more of a natural or synthetic biodegradable material.

17. The method of claim 12 wherein the wall of the insertion guide is formed of a one or more of a bioabsorbable, biodegradable or bioerodable polymer.

18. The method of claim 12 wherein the insertion guide substantially degrades in a few hours.

19. The method of claim 12 wherein the insertion guide substantially degrades in 1-3 days.

20. The method of claim 12 wherein the insertion guide wall is formed of a polymer in the form of a mesh.

21. A system for positioning a medical device in a brain comprising:
an insertion guide formed of a sleeve having a lumen therein and extending along the length thereof, the sleeve being tubular in shape and formed of a biodegradable material; and
a rod configured to be disposed coaxially within said sleeve and wherein a portion of the length of said rod extends outwardly from the proximal end of said sleeve, the rod for providing a desired stiffness to the combination sleeve and rod for insertion into the brain and configured to be removed from the sleeve after the combination rod and sleeve reaches a desired position in the brain, the sleeve configured to remain in place and provide a path way for the medical device to be inserted into the desired position in the brain.
